(19) United States
(12) Patent Application Publication
(10) Pub. No.: US 2007/0269513 A1
(43) Pub. Date: Nov. 22, 2007

(54) Liver selective drug therapy

(76) Inventor: Howard John Smith, Templestone (AU)

Correspondence Address: BIO INTELLECTUAL PROPERTY SERVICES (BIO IPS) LLC, 8509 KERNON CT., LORTON, VA, 22079, US

(21) Appl. No.: 11/819,278

(22) Filed: Jun. 26, 2007

Related U.S. Application Data
(63) Continuation-in-part of application No. 10/137,444, filed on May 3, 2002, now abandoned.
Application No. 10/137,444 is a continuation-in-part of application No. PCT/AU00/01337, filed on Nov. 1, 2000.

(30) Foreign Application Priority Data
Feb. 7, 2000 (AU) PQ5471
Nov. 3, 1999 (AU) PQ3855
Jan. 24, 2000 (AU) PQ5236

(51) Int. Cl. A61K 9/22; A61K 31/21; A61P 1/16

(52) U.S. Cl. 424/468; 514/510

(57) ABSTRACT

A method of pharmaceutical therapy comprising administering a pharmaceutical complementary medicine or herbal product orally at a dose sufficient to provide a clinically effective level in the portal vein and less than that required to provide a clinically effective blood level in the peripheral circulation to thereby provide a dose-delivery rate having a selective clinical effect in the liver.

### CROSS-REFERENCE TO RELATED APPLICATIONS

This application is a continuation-in-part of U.S. application Ser. No. 10/137,444, filed May 3, 2002, which is continuation-in-part of application No. PCT/AU00/01337, filed on Nov. 1, 2000, which claims benefit of AU PQ3855 filed on Nov. 3, 1999; AU PQ5236 filed on Jan. 24, 2000, and AU PQ5471 filed on Feb. 7, 2000. The entire disclosure of these prior applications are hereby incorporated by reference.

### STATEMENT REGARDING FEDERALLY SPONSORED RESEARCH OR DEVELOPMENT

This invention has been created without the sponsorship or funding of any federally sponsored research or development program.

### SEQUENCE LISTING OR PROGRAMS

Not Applicable

### BACKGROUND OF THE INVENTION—FIELD OF THE INVENTION

The present invention relates to a method of drug treatment where the liver or portal venous circulation is the primary therapeutic target, and in particular to a method of treatment or prevention of diseases, that is selective for the liver and thereby minimizes side effects. Also included are the treatment of systemic diseases where the therapeutic management is directed towards a physiological or disease process acting within the liver itself.

### BACKGROUND

The traditional methods of oral therapy for management of any disease usually require a drug to be administered by mouth to reach systemic levels of active agent within the body and circulation and to achieve the desired therapeutic effect. Since all substances absorbed from the gastrointestinal tract are then released into the portal venous circulation, they must then pass through the liver before entering the systemic circulation. The liver is generally and correctly perceived as an obstruction to the systemic bioavailability of a drug because many substances are excreted from the body through hepatic metabolism. The phenomenon of rapid uptake followed by metabolism of drugs during their first exposure to the liver is known as first-pass clearance by the liver. This phenomenon of first-pass clearance, together with later uptake and metabolism during subsequent transits of the liver is the principal cause of short half-life of a drug. The problem of short half-life may be addressed by 1) using loading doses of a drug to ensure that adequate systemic levels are achieved, 2) administering a drug several times a day, 3) by administering the drug by a different route, for example parenterally or transdermally, or 4) by developing medicines that are not taken up or metabolised by the liver, and hence have long half-life.

Many of the statin drugs which exhibit a short half-life, such as simvastatin and fluvastatin, have a short half-life because of their metabolism by the liver. All molecules like this have a modest degree of liver-selectivity, but the fact remains that the same molecules can and do produce side effects elsewhere in the body. Therefore there is an opportunity and desirability to increase the relative liver-selectivity of short acting statins and thereby reduce the presence and incidence of side effects throughout the body.

As the description of the present invention describes, the relative liver-selectivity of short-acting statins can be increased by administering them at both at a low dose and in a slow-release formulation. When a drug is administered in this way, therapeutic concentrations of the drug within the liver are maintained for longer periods of time by the slow release of drug from the formulation and the continuous uptake of the drug into the portal venous system. By contrast, when a drug is administered in a conventional formulation, therapeutic concentrations within the liver are maintained by direct absorption into the portal venous system plus the continuous perfusion of the liver from the systemic circulation.

The ability to increase the relative selectivity of short-acting statins by presenting them to the body in a low dose, slow-release formulation does not apply to longer acting statins since these pass on into the systemic circulation and thence pass through the liver again and again. For almost all drugs, a short half life is usually regarded by medical practitioners and drug developers as a weakness, however it becomes a strength if presentation at low dose, and in a sustained release formulation, achieves or increases therapeutic effectiveness as with the selective delivery of the short half-life statins to the liver.

The specific pharmacological action of statins is inhibition of the enzyme HMG CoA reductase. Within the liver, this enzyme is a key step in the synthesis of cholesterol. Therefore, inhibition of HMG CoA reductase inhibits the synthesis of cholesterol.

The same enzyme is also a key step in the synthesis of coenzyme Q10 (also known as ubiquinone) in every cell of the body. Coenzyme Q10 is found in mitochondria and is required to facilitate production of cell energy as ATP. Systemic inhibition of the enzyme causes reduction in coenzyme Q10, and this results in undesirable side effects.

Side effects caused by statins, and related to inhibition of synthesis of ubiquinone are found principally in skeletal muscle, but may also occur in cardiac muscle and the brain.

Side Effects of Statins in Skeletal Muscle.

The product information for all statins invariably notes that these drugs may cause side effects in skeletal muscle.

Simvastatin

The product information for simvastatin in USA and Australia (attached) highlights the very low risk of myopathy/rhabdomyolysis on monotherapy with simvastatin, but notes that the rate may increase when the drug is given with other lipid-lowering drugs. The simvastatin Product Information notes that myopathy/rhabdomyolysis may be diagnosed by noting symptoms and measuring the plasma creatine kinase levels.

The detailed advice given in the Product Information for simvastatin is a recognition by the manufacturer that significant effects may occur beyond the liver.

Other Statins

The Product Information for all other statins contains a warning about the risk of myopathy.

Rosuvastatin (Astra Zeneca)

The Product Information for rosvastatin in Australia notes that unexplained muscle aches, mild to severe pain or stiffness and weakness may even occur when creatine kinase levels are normal.

The Rosuvastatin PI States

“Statins pose a risk of myopathy and rhabdomyolysis Stop treatment with rosuvastatin if patients develop unexplained muscle aches, mild to severe pain, or stiffness or weakness, even when plasma creatine kinase levels are normal. Monitor creatine kinase at baseline and repeat during treatment if clinically indicated or with any increase in dose.”

Given the appropriately high rate of treatment with cholesterol-lowering drugs in cardiac patients, it is also very possible that the incidence of muscle problems in patients on statins is under diagnosed when fatigue and/or weakness is attributed to accompanying cardiac disease.

Baycol

In 2001 Bayer Pharmaceutical Division withdrew (Baycol) cerivastatin from development because of the incidence of severe and sometimes fatal rhabdomyolysis

The public statement by the FDA read as follows:

Aug. 8, 2001 FDA today announced that Bayer Pharmaceutical Division is voluntarily withdrawing Baycol (cerivastatin) from the U.S. market because of reports of sometimes fatal rhabdomyolysis, a severe muscle adverse reaction from this cholesterol-lowering (lipid-lowering) product.

Cardiac Myopathy

There are some reports that statins may produce changes in cardiac muscle similar to their effects in skeletal muscle, but the situation is less clear. Other reports have suggested that these drugs may have beneficial effects on muscle growth within the heart. Either event would be consistent with a systemic effect of the drug beyond the liver.

This is an area that is continuing to attract research, nevertheless is it is a not uncommon practice to stop treatment with statins before elective general or cardiac surgery to avoid the possible risk of depressed function of skeletal or cardiac muscle.

Transient Global Amnesia

In recent years there have been a growing number of reports linking statins to Transient Global Amnesia.

Lay Reports

Most of these reports have appeared in the lay press, rather than in documents supported by or published by industry. Prominent among these is the book by Duane Graveline MD entitled LIPITOR Thief of memory (published by Infinity Publishing Com., Haverford, Pa. 19041-1413, January 2004).

Dr Graveline describes his own personal experiences of statin-induced transient global amnesia, together with many other cases reported by patients and their physicians. He concludes that this problem may be more common. with lipophilic statins such as atorvastatin, lovastatin, and simvastatin (p 34).

In his book, Dr Graveline also reviews the more widely accepted phenomenon of statin-induced rhabdomyolysis and notes that some cases of cardiomyopathy may also be linked to the use of statins.

While Dr Graveline is more well known as a retired astronaut, the foreword of Dr Graveline's book is written by the eminent Professor Jay S Cohen of University of California San Diego. He writes, “I knew that statin drugs like Lipitor could cause cognitive, emotional, and memory problems, but I didn't know they could cause amnesia.”

and

“So-called minor side effects are also commonly overlooked in pre-approval studies, so these side effects aren't listed in package inserts or the Physicians' Desk Reference.”

Regulatory Reports

Transient global amnesia has been monitored by the National Prescribing Service in Australia.

Their review as at 18 Nov. 2004 can be retrieved at www.nps.org.au/resources/content/factsheet_statins_and_memory_loss.pdf

The Australian National Prescribing Service noted the following cases within Australia:
Atorvastatin 7 cases
Fluvastatin 1 case
Pravastatin 3 cases
Simvastatin 22 cases

The review by The Australian National Prescribing Service also cites the American report authored by Wagstaff and colleagues that has reviewed 60 reports notified to the Medwatch, the Drug Surveillance system of the FDA. (Wagstaff L R and others. Statin-associated memory loss: analysis of 60 case reports and review of the literature. Pharmacotherapy 23 (7), 871 - 880, 2003.)

The purpose of including these references to Transient Global Amnesia in this document is simply to note that statins as a group of drugs can have side effects in the body that are independent of the effect of the drug on the liver. Therefore there is an opportunity to increase the relative liver-selectivity of the drugs so that their direct hepatic therapeutic effect is maintained, but the risk of peripheral or systemic side effects is reduced to even lower or to zero levels.

Product Information Warnings on Myopathy/Rhabdomyolysis

A. Warnings listed in the USA—Approved Prescribing Information for Zocor (Simvastatin)

Warnings

Myopathy/Rhabdomyolysis

Simvastatin, like other inhibitors of HMG-CoA reductase occasionally causes myopathy manifested as muscle pain, tenderness or weakness with creatine kinase (CK) above 10× the upper limit of normal (ULN). Myopathy sometimes takes the form of rhabdomyolysis with or without acute renal failure secondary to myoglobinuria, and rare fatalities have occurred. The risk of myopathy is increased by high levels of HMG CoA reductase inhibitory activity in the plasma.

As with other HMG-CoA reductase inhibitors, the risk of myopathy/rhabdomyolysis is dose related. In a clinical trial base in which 41,050 patients were treated with Zocor with 24,474 (approximately 60%) treated for at least 4 years, the incidence of myopathy was approximately 0.02%, 0.08% and 0.53% at 20, 40 and 80 mg/day respectively. In these trials, patients were carefully monitored and some interacting medicinal products were excluded.

All patients starting therapy with simvastatin, or whose dose of simvastatin is being increased, should be advised of the risk of myopathy and told to report promptly any unexplained muscle pain, tenderness or weakness. Simvastatin should be discontinues immediately if myopathy is diagnosed or suspected. In most case muscle symptoms and CK increases resolved when treatment was promptly discontinued. Periodic CK determinations may be considered in patients starting therapy with simvastatin or whose dose is being increased, but there is no assurance that such monitoring will prevent myopathy.

Many of the patients who have developed rhabdomyolysis on therapy with simvastatin have complicated medical histories, including renal insufficiency usually as a consequence of longstanding diabetes. Such patients merit closer monitoring. Therapy with simvastatin should be temporarily stopped a few days prior to elective major surgery and when any major medical or surgical condition intervenes.

The risk of myopathy/rhabdomyolysis is increased by concomitant use of simvastatin with the following:

Potent inhibitors of CYP3A4 Simvastatin, like several other inhibitors of HMGCoA reductase is a substrate of cytochrome P450 3A4 (CYP3A4). When simvastatin is used with a potent inhibitor of CYP3A4, elevated plasma levels of HMG CoA reductase inhibitory activity can increase the risk of myopathy and rhabdomyolysis, particularly with higher doses of simvastatin.

The use of simvastatin concomitantly with the potent CYP3A4 inhibitors itraconazole, ketoconazole, erythromycin, clarithromycin, telithromycin, HIV protease inhibitors, nefazodone, or large quantities of grape fruit juice (>1 quart daily) should be avoided. Concomitant use of other medicines labelled as having a positive effect on CYP3A4 unless the benefits of combined therapy outweigh the risk. If treatment with itraconazole, ketoconazole, erythromycin, clarithromycin, or telithromycin is unavoidable, therapy with simvastatin should be suspended during the course of treatment.

Gemfibrozil, particularly with higher doses of simvastatin: The dose of simvastatin should not exceed 10 mg daily in patients receiving concomitant medication with gemfibrozil. The combined use of simvastatin and gemfibrozil should be avoided unless the benefits are likely to outweigh the increased risks of this combination.

Other lipid-lowering drugs (other fibrates or

$$1 { g}_{\geq } / day of niacin :$$

Caution should be used when prescribing other fibrates or lipid lowering doses

$$1 g / {d}^{\geq } ay ) {of}^{\geq }$$

niacin with simvastatin as these agents can cause myopathy when given alone. The benefit of further alterations in lipid levels by the combines use of simvastatin with other fibrates or niacin should be carefully weighed against the potential risks of these combinations.

Cyclosporin or danazole, with higher doses of simvastatin: The dose of simvastatin should not exceed 10 mg daily in patients receiving concomitant medication with cyclosporin or danazole. The benefits of the use of simvastatin in patients receiving cyclosporin or danazole should be carefully weighed against risks of these combinations.

Amiodarone or verapamil with higher doses of simvastatin: The dose of simvastatin should not exceed 20 mg daily in patients receiving concomitant medication with amiodarone or verapamil. The combined use of simvastatin at doses higher than 20 mg daily with amiodarone or verapamil should be avoided unless the clinical benefit is likely to outweigh the increased risk of myopathy. In an ongoing clinical trial, myopathy has been reported in 6% of patients receiving simvastatin 80 mg and amiodarone. In an analysis of clinical trials involving 25,248 patients treated with simvastatin 20 to 80 mg, the incidence of myopathy was higher in patients receiving verapamil and simvastatin (4/635; 0.63%), than in patients taking simvastatin without a calcium channel blocker (13/21,224; 0.061%).

B. Warnings listed in the Australian—Approved Product information Zocor (Simvastatin)

Merck Sharp and Dohme

Precautions

Use with caution in the following circumstances

Simvastatin and other inhibitors of HMG-CoA reductase occasionally cause myopathy manifested as muscle pain, tenderness or weakness with creatine kinase (CK) above 10× the upper limit of normal (ULN). Myopathy sometimes takes the form of rhabdomyolysis with or without acute renal failure secondary to myoglobinuria, and rare fatalities have occurred. The risk of myopathy is increased by high levels of HMG CoA reductase inhibitory activity in the plasma. The risk of myopathy/rhabdomyolysis is increased by concomitant use of simvastatin with the following:
Potent inhibitors of CYP3A4 Cyclosporin, itraconazole, ketoconazole, erythromycin, clarithromycin, HIV protease inhibitors or nefazodone, particularly with higher doses of simvastatin (see Interactions with other drugs, CYP3A4 interactions and Pharmacology, Pharmacokinetics).
Lipid lowering drugs that can cause myopathy when given alone.

Gemfibrozil, other fibrates or lipid lower doses (greater than or equal to 1 g/day) of niacin, particularly with higher doses of simvastatin (see Interactions with other drugs, Interactions with lipid lowering drugs that can cause myopathy when given alone and Pharmacology, Pharmacokinetics).
Other drugs.

Amiodarone or verapamil with higher doses of simvastatin (See Interaction with other drugs, Other drug interactions.

The risk of myopathy/rhabdomyolysis is dose-related. The incidence in clinical trials in which patients were carefully monitored and some interacting drugs excluded, has been approximately 0.03% at 20 mg, 0.08% at 40 mg and 0.4% at 80 mg.

Consequently:
1. Use of simvastatin with itraconazole, ketoconazole, erythromycin, clarithromycin, HIV protease inhibitors or nefazodone should be avoided. If treatment with itraconazole, ketoconazole, erythromycin, clarithromycin, HIV protease inhibitors or nefazodone is unavoidable, therapy should be suspended during the course of treatment. Concomitant use with other medicines labelled as having a potent effect on CYP3A4 at therapeutic doses should be avoided unless the benefits of combined therapy outweigh the increased risk.
2. The dose of simvastatin should not exceed 10 mg daily on patients receiving concomitant medication with cyclosporine, gemfibrozil, other fibrates or lipid lowering doses (greater than or equal to 1 g/day) of niacin. The combined use of simvastatin with fibrates or niacin should be avoided unless the further benefit of further alteration in lipid levels is likely to outweigh the increased risk of this drug combination.
3. The dose of simvastatin should not exceed 20 mg/day in patients receiving amiodarone or verapamil. The combined use of simvastatin at doses higher than 20 mg daily with amiodarone or verapamil should be avoided unless the clinical benefit is likely to outweigh the increased risk of myopathy.
4. All patients starting therapy with simvastatin, or whose dose of simvastatin is being increased, should be advised of the risk of myopathy and told to report promptly any unexplained muscle pain, tenderness or weakness. Simvastatin should be discontinues immediately if myopathy is diagnosed or suspected. The presence of these symptoms and a CK >10 times the upper limit of normal indicates myopathy. In most case when patients were promptly discontinued from treatment muscle symptoms and CK increases resolved. Periodic CK determinations may be considered in patients starting therapy with simvastatin or whose dose is being increased, but there is no assurance that such monitoring will prevent myopathy.
5. Many of the patients who have developed rhabdomyolysis on therapy with simvastatin have complicated medical histories, including renal insufficiency usually as a consequence of longstanding diabetes. Such patients merit closer monitoring. Therapy with simvastatin should be temporarily stopped a few days prior to elective major surgery and when any major medical or surgical condition intervenes.

C. Similar warnings can be found in the approved product information for other statins.

| | | |
|---|---|---|
| Atorvastatin | Pfizer | www.lipitor.com |
| Fluvastatin | Novartis | www.novartis.us.com/product |
| Lovastatin | Merck | www.merck.com/product |
| Pravastatin | Squibb | www.rzlist.com/cgi/generic/pravast/hotmail |
| Rosuvastatin | Astra-Zeneca | www.crestor.info |

D. JAMA. 2003 Apr 2; 289(13):1681-90

Statin-associated myopathy.

Thompson P D, Clarkson P, Karas R H.

Preventive Cardiology and Cardiovascular Research, Division of Cardiology, Hartford Hospital, Hartford, Conn. 06102, USA.

Statins (3-hydroxy-3-methylglutaryl coenzyme A reductase inhibitors) are associated with skeletal muscle complaints, including clinically important myositis and rhabdomyolysis, mild serum creatine kinase (CK) elevations, myalgia with and without elevated CK levels, muscle weakness, muscle cramps, and persistent myalgia and CK elevations after statin withdrawal. We performed a literature review to provide a clinical summary of statin-associated myopathy and discuss possible mediating mechanisms. We also update the US Food and Drug Administration (FDA) reports on statin-associated rhabdomyolysis. Articles on statin myopathy were identified via a PubMed search through November 2002 and articles on statin clinical trials, case series, and review articles were identified via a PubMed search through January 2003. Adverse event reports of statin-associated rhabdomyolysis were also collected from the FDA MEDWATCH database. The literature review found that reports of muscle problems during statin clinical trials are extremely rare. The FDA MEDWATCH Reporting System lists 3339 cases of statin-associated rhabdomyolysis reported between Jan. 1, 1990, and Mar. 31, 2002. Cerivastatin was the most commonly implicated statin. Few data are available regarding the frequency of less-serious events such as muscle pain and weakness, which may affect 1% to 5% of patients. The risk of rhabdomyolysis and other adverse effects with statin use can be exacerbated by several factors, including compromised hepatic and renal function, hypothyroidism, diabetes, and concomitant medications. Medications such as the fibrate gemfibrozil alter statin metabolism and increase statin plasma concentration. How statins injure skeletal muscle is not clear, although recent evidence suggests that statins reduce the production of small regulatory proteins that are important for myocyte maintenance.

### SUMMARY OF THE INVENTION

In accordance with the present invention we provide a method of pharmaceutical therapy comprising administering a pharmaceutical orally at a dose sufficient to provide a clinically effective level in the portal vein and liver, but less than that required to provide a clinically effective blood level in the peripheral circulation. The method thereby provides a dose-delivery rate with a clinically-selective effect in the liver.

Diseases of the liver and portal circulation to which this invention applies include portal hypertension resulting from cirrhosis of the liver, hypercholesterolaemia, viral hepatitis of any form including hepatitis A, B, C, D, E, G, and other viral infections, autoimmune hepatitis, hepatic hypoxic conditions resulting from primary disease of the liver or secondary to extrahepatic diseases, and any other condition where the liver itself is the primary therapeutic target and it is desirable to concentrate a therapeutic agent within the liver.

The present invention provides a method of administering a drug with an intrinsic short half-life, at a low dose, and in a slow-release formulation. In this way, clinically effective concentrations of a drug will be achieved in the portal circulation and within the liver itself. However, clinically effective blood levels will not be achieved in the peripheral or systemic circulation because 1) a significant portion of the drug is removed by the liver during first-pass, and 2) the relatively large volume of the systemic circulation compared with the smaller volume portal circulation creates a dilution effect. It is therefore an underlying principle of this invention that short half-life of a drug becomes a strength rather than a weakness, and can be employed to achieve relative selectivity of a therapeutic effect.

The principles of liver-selective delivery of apply to any condition where the liver or portal venous circulation is the primary target for drug treatment. Many diseases are presently treated with systemic doses of established drugs, or are intended to be treated with novel classes of drugs presently in development. It is a key principle of this invention that the use of low-dose, slow-release formulations of these drugs will achieve the desired therapeutic effect in a manner similar to, or more effective than present treatment, but with a much lower rate of systemic side effects. Thus, the use of liver-selective delivery of drugs for treatment of liver disease can expect a greater tolerance, acceptance and compliance by patients.

The method of the invention involves the oral administration of a pharmaceutical at a dose-delivery rate sufficient to provide a clinically effective blood level in the portal system but less than that required to provide a clinically effective blood level in the peripheral circulation. The dose-delivery rate is typically achieved by a slow release formulation.

The principle of achieving liver-selectivity by use of a slow release formulation also applies to the use of a slow infusion of a medicine into the gastrointestinal tract through a naso-gastric tube or other artificial access. While such a route of administration will usually be impracticable for chronic treatment, the use of this technique in situations of acute medical care may ensure delivery of a therapeutic agent to the body, and at the same time minimise systemic side effects.

The principle of liver-selective delivery of drugs can be described mathematically in the following way.

Consider a drug administered by mouth as a slow-release formulation to achieve steady state release into the bowel with uptake into the portal venous circulation. The drug is then partly metabolised by the liver.

Let the volume of blood passing through the portal circulation in unit time=V_{P} litres.

Let the total volume of the systemic circulation=V_{S} litres.

Let the concentration of drug in the portal vein=C_{P} mg/litre.

Let the concentration of drug in the systemic circulation=C_{S} mg/litre.

Drug absorbed from the GI tract in unit time−D_{A} mg.

Drug metabolised by the liver in unit time=D_{M} mg

Drug not metabolised by the liver in unit time=D_{A}−D_{M} mg=D_{NM} mg

Let the metabolic clearance=M

This must range from 0 (no clearance) to 1.0 (total clearance).

Then C_{P} is determined by the amount of drug absorbed into the finite V_{P} plus the concentration in the drug recirculated.
C_{P}=D_{A}/V_{P}+C_{S}
i.e.,
D_{A}=V_{P}(C_{P}−C_{S}) equation. 1

Drug metabolised is a function of clearance rate, portal venous concentration and portal volume per unit time.
D_{M}=M×C_{P}×V_{P} equation. 2

Systemic concentration of drug is determined by the volume of the systemic circulation and the amount of drug not metabolised
C_{S}=D_{NM}/V_{S}
i.e.,
D_{NM}=C_{S}×V_{S} equation. 3

By definition,
D_{A}=D_{M}+D_{NM}

Substituting equations 1,2, and 3,
V_{P} (C_{P}−C_{S})=M×C_{P}×V_{P}+C_{S}×V_{S}
and
C_{P}[V_{P}(1−M)]=C_{S}(V_{S}+V_{P})
such that
C_{P}/C_{S}=(V_{S}+V_{P})/V_{P}(1−M)

This relationship may be interpreted in the following way.

1. When there is no metabolic clearance of a drug by the liver, (M=0), the concentration gradient between portal and systemic vessels during steady state release of a drug from a slow-release formulation is a function of their relative volumes of the two circulations. C_{P}/C_{S}=(V_{S}+V_{P})/V_{P}

2. With total hepatic clearance, M=1, and C_{P}/C_{S} tends towards infinity.

3. If the rate of metabolism by the liver saturates, M will decline at higher dose levels. Therefore liver selectivity will be greater at lower dose levels, and be maximal when there is no effective saturation of metabolism.

4. Portal venous flow does vary. Therefore C_{P}/C_{S} will be higher under low-flow conditions, for example in cirrhosis, but be low in high-flow situations such as when there is an abnormal shunting of blood perhaps through fistulae.

Treatment of Portal Hypertension

A specific aspect example of liver-selective delivery is liver-selective beta-blockade for the treatment of portal venous hypertension. The present invention therefore relates to a method of treatment of portal hypertension and prevention of variceal bleeding.

Portal hypertension is a common complication of cirrhosis of the liver and is defined by the elevation of venous pressure in the portal vein to levels >30 cm saline.

The portal vein is the final common conduit for blood draining the major part of the gastrointestinal tract including stomach, and both the small and large bowel, and passing to the liver. Because the vein lacks valves, any obstruction to the flow of blood within the liver, within the portal vein itself, or by elevation of pressure in the inferior vena cava, causes elevation of the pressure in the portal vein and its tributaries. In practice, the most common cause of portal hypertension is cirrhosis of the liver, of which the most common cause is end-stage alcoholic liver disease. In the USA, clinically significant portal hypertension is present in more than 60% of patients with cirrhosis.

The symptoms of portal hypertension are usually superimposed on the symptoms of the underlying liver disease and impaired liver function. They include the physical effects of raised portal vein pressure—haemorrhage from gastro-oesophageal varices (variceal bleeding), splenomegaly with hypersplenism, and ascites, which is fluid leak into the peritoneal space. Acute haemorrhage into the bowel from bleeding varices is the most serious complication, and may produce acute shock and death. It is therefore a life-threatening emergency. Milder cases of haemorrhage may present as melena, which is usually interpreted as a warning of potential massive haemorrhage.

The treatment of variceal bleeding includes conventional methods of blood and fluid replacement to restore blood volume and pressure. In addition, local treatment with balloon tamponade, sclerosis of varices and selected vasoconstrictors may be employed.

Prevention of variceal bleeding utilises techniques that can lower portal venous pressure and thereby reduce the chance of rupture. Several surgical techniques have been developed but these are by their nature invasive. An alternative method has been to administer beta-adrenergic antagonists (beta-blockers) particularly propranolol. Beta-blockers inhibit the action of the beta-adrenergic effect of adrenaline throughout the body, including the constrictor effect of adrenaline on the portal vein. Therefore, they act to lower portal venous pressure, and have been shown to prevent a first variceal bleed and subsequent episodes after an initial bleed.

The use of beta-blockers such as propranolol in patients with portal hypertension and advanced liver disease has up until now not been widely accepted because the systemic effects of the drug are cardiac with potential adverse effects in these patients. Beta-blockers slow the heart, lower blood pressure, and may mask the early signs of shock in a patient who is bleeding internally. Beta-blockers frequently cause both fatigue and lethargy, which are common symptoms in patients with liver disease. Since propranolol is also metabolised by the liver, the inability of an impaired liver to clear the drug from the circulation when the drug is given in normal systemic doses may cause plasma levels to rise thereby exacerbating cardiac symptoms, and in severe cases precipitate encephalopathy.

Therefore, while current medical textbooks note the potential of propranolol to lower portal venous pressure and reduce variceal haemorrhage, the prescribing information for propranolol in most countries specifically warns against the use of the drug in patients with decompensated cirrhosis, noting that encephalopathy may develop and symptoms of haemorrhage may be masked.

In this first aspect of the invention, we provide a method of treatment of portal hypertension including the administration of propranolol in a form selective for the liver that will reduce portal venous pressure with minimal risk of adverse systemic effects. The method involves use of a slow-release formulation of a low dose of a beta-blocker such as propranolol, being a drug that is metabolised by the liver with relatively high first pass clearance. In this way, clinically effective blood levels of the drug will be achieved in blood reaching the liver and the portal circulation, but not the peripheral blood circulation.

In the treatment of portal hypertension, the primary target of the beta-blocker drug is the portal circulation, that is, at a circulatory level before the drug is cleared from the circulation by the liver. Therefore, the requirement is for effective plasma concentrations of the drug in blood that has not yet passed through the liver. This is in contrast to the treatment of cardiac conditions where a drug must clear first-pass metabolism by the liver and then disperse throughout the much larger systemic blood volume. Therefore, when a drug is given as a low-dose sustained release formulation, effective plasma concentrations of the drug will be achieved in the portal circulation at lower daily doses than are required to achieve systemic effects. Two other features of cirrhosis with portal hypertension also act to reduce the rate of drug metabolism by the liver. Impaired liver function itself reduces drug clearance and venous obstruction reduces portal blood flow. This means that the daily dose of slow-release of formulation of propranolol required to achieve clinically useful blood levels in the portal circulation may be as low as one tenth to one twentieth of those required to achieve systemic effects, for example, the doses used to treat cardiac disease. Thus, while the dose of propranolol used in systemic doses to treat portal hypertension is in the range of 80-160 mg or more per day, the dose used as a liver-selective formulation will be in the range 10-25 mg per day. The daily dose will be least in those patients with the most severe cirrhosis of the liver because very slow portal venous blood flow is a feature of this condition. In any patient, the optimum dose should be the highest dose that does not produce evidence of systemic beta-blockade as evidenced by inhibition of tachycardia.

Preferred compounds are beta-adrenergic antagonists (beta-blockers) that are non-selective (having both beta-1 and beta-2 properties), and are metabolised by the liver. This includes almost all lipophilic beta-blockers including propranolol, nadolol, oxprenolol, and other compounds. These compounds have a short half-life, where the half-life is a function of metabolism by the liver. This is contrary to the discipline of drug development, which has, where possible, selected agents with longer half-lives to allow once-a-day administration. In the present invention, the slow-release formulation enables a continuous low dose to be delivered to the liver and the portal circulation, and achieve therapeutic levels, without reaching clinically significant levels in the peripheral circulation.

Treatment of Hypercholesterolaemia

The present invention also relates to a method of treatment of hypercholesterolaemia and in particular to a method of treatment of hypercholesterolaemia using HMG-CoA reductase inhibitors such as the statin class of drugs, being compositions containing HMG-CoA reductase inhibitors.

Atherosclerosis and its various clinical presentations as coronary artery disease, cerebrovascular disease, peripheral vascular disease and other conditions, is a major cause of death in western countries. Hypercholesterolaemia is a primary risk factor for death from these conditions. HMG-CoA reductase (3-hydroxy-3-methyl-glutaryl-coenzyme A) inhibits the rate determining step in cholesterol biosynthesis (conversion of HMG-CoA to mevalonate), and inhibitors of HMG-CoA reductase have proved to be most effective in reducing the plasma levels of cholesterol in patients with both hypercholesterolaemia and normocholesterolaemia. For example, simvastatin (also known as lovastatin) in clinical trials reduced cholesterol and LDL cholesterol by 25% and 35% respectively. Simvastatin was reported in trials to reduce the risk of a major coronary event by 34%.

The statins have been effectively used in treating individuals with high cholesterol for many years. However the treatment of patients with inhibitors of HMG-CoA reductase such as the statins is accompanied by adverse side effects which cause discomfort and may necessitate discontinuation of medication. As HMG-CoA reductase inhibitors are used as a long term means for prevention of heart disease in patients who may be otherwise healthy, there is a need for a method of treatment of hypercholesterolaemia without the associated effects of HMG-CoA reductase inhibitors.

Adverse effects known to be associated with the use of HMG-CoA reductase inhibitors include muscle cramps, myalgia, increased risk of myopathy, transient elevation of creatine phosphokinase levels from skeletal muscle, and even rhabdomyolysis. The risk of these side effects is further increased when some other lipid lowering drugs, particularly gemfibrizol are coprescribed.

The use of HMG-CoA reductase inhibitors has also been reported to aggravate cardiac function, and (uncommonly) to worsen cardiac failure. These adverse effects in both skeletal muscle and the heart, are not common, but appear to have a common pathway related to inhibition of the synthesis of ubiquinone.

HMG-CoA reductase is a key enzyme in the synthesis of ubiquinone (also known as coenzyme Q10), because this substance is also synthesised from mevalonate. Therefore, HMG-CoA reductase inhibitors cause depletion of coenzyme Q10. The role of HMG-CoA reductase in synthesis of ubiquinone and cholesterol may be schematically shown as follows:

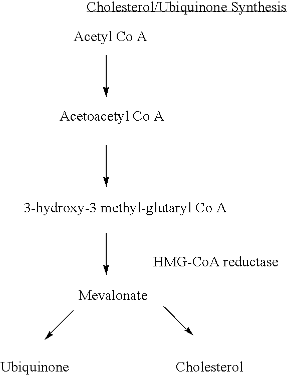

Coenzyme Q10 is a key redox coenzyme of the respiratory chain responsible for energy production within mitochondria throughout the body. These processes have been termed “bioenergetics”. Depletion of Coenzyme Q10 in skeletal and cardiac muscle has been linked to the development of both skeletal myopathy and cardiac myopathy, to the development of fatigue, and has been proposed as the mechanism of action of statin-induced muscle disease. Since fatigue is a widely-reported symptom in patients with cardiovascular disease, many of whom are taking HMG-CoA reductase inhibitors for treatment of hypercholesterolaemia, it is likely that a contribution to the cause of fatigue by these drugs has not been appreciated and therefore under diagnosed.

U.S. Pat. No. 5,316,765 describes a method and composition for reducing the side effects of HMG Co A reductase inhibitors, which involves concurrent administration of coenzyme Q10 in an attempt to offset the clinical effects of inhibiting formation of coenzyme Q10.

Reports published in the scientific literature attest the use in selected patients of dietary ubiquinone to reverse clinically significant adverse effects of HMG Co A reductase inhibitors in skeletal muscle or presenting as cardiac dysfunction.

In this second aspect of the invention, we provide a method of treatment of hypercholesterolaemia including administration of an HMG CoA reductase inhibitor in a form selective for the liver, that will reduce hypercholesterolaemia without systemic depression of Coenzyme Q10 and its sequelae of muscle disease and other conditions including the heart. The method involves use of a slow-release formulation of a low dose of HMG CoA reductase inhibitor that is itself metabolised by the liver. In this way, clinically effective blood levels of the HGM Co A reductase inhibitor will be achieved in blood reaching the liver through the portal venous system, but not in the peripheral blood circulation.

As 90% of cholesterol synthesis within the body occurs in the liver but ubiquinone synthesis is a systemic cell process the method of the invention provides effective cholesterol control without the same risk of side effects associated with previous treatments.

Preferred compounds are simvastatin (also known as lovastatin), pravastatin, mevastatin and atorvastatin. The invention may however be applied to any lipid-lowering agent that also depresses levels of ubiquinone (coenzyme Q10). Other examples of other compounds include fibrates such as gemfibrizol. Preferred compounds will be absorbed from all or almost all of the small bowel, and have a short half-life on account of metabolism by the liver. It is likely that such a compound will be lipophilic.

The preferred statin type HMG-CoA reductase inhibitors generally have their formulas: 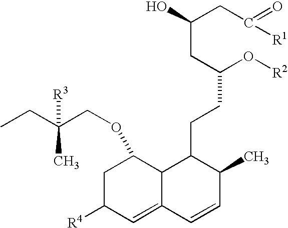
Wherein
R¹ is OR⁵ wherein R⁵ is a counter ion such as sodium
R³ is a hydrogen or methyl.
R⁴ is selected from hydrogen, hydroxy and methyl,
R² is hydrogen or R¹ and R² may together form a bond to provide a lactone.

In a further aspect, the invention provides for the use of an HMG CoA-reductase inhibitor formulated as a slow-release pharmaceutical for treatment or prophylaxis of hypercholesterolaemia.

Formulations that release the HMG-CoA reductase inhibitor slowly over 24 hours (permitting once a day administration by mouth) or over 12 hours (permitting twice a day administration by mouth) will effectively control plasma cholesterol without the need to expose the peripheral circulation to active levels of the drug. The release characteristics of the slow-release formulation will provide a daily dosage of the HMG-CoA reductase inhibitor at less than the dose of the drug when used in full clinical or systemic doses as a conventional formulation.

The differences between the kinetics of HMG CoA Reductase Inhibitors as a class and the beta-adrenergic antagonist propranolol need to be noted. In contrast to propranolol, simvastatin is known to have very high first pass clearance by the liver—up to 92%. This means that in contrast to propranolol, simvastatin is inherently liver-selective without the need for special formulation. However simvastatin has a half-life of 0.7-4.0 hours so that full doses are required to achieve a clinically effective cholesterol lowering effect. Furthermore the exposure to the rest if the body of the 8% of drug that is not cleared by the liver appears to be sufficient to produce adverse events in some people. It is the claim of this invention that presentation of HMG CoA Reductase inhibitors as slow-release and low-dose formulations will reduce or permit avoidance of all adverse events associated with systemic depletion of ubiquinone. At the same time, delivery of an HMG CoA Reductase Inhibitor as a slow-release formulation will lower plasma cholesterol in a manner similar to or greater than systemic doses in conventional formulations.

In the case of simvastatin, which is normally given in doses of 20-80 mg per day, a liver selective formulation presented as a slow-release formulation will be less. The final doses required will need to be established in clinical trials but may be in the range 5-20 mg per day.

The most preferred HMG-CoA reductase inhibitors for use in the invention are those with a short half-life, where the short half-life is a function of metabolism by the liver. This is contrary to the discipline of drug development, which has, where possible, selected agents with longer half-lives to allow a once a day administration. In the present invention, the slow-release formulation enables a continuous low dose to be delivered to the liver and achieve therapeutic levels within the liver, without reaching clinically significant levels in the peripheral circulation.

Autoimmune Hepatitis

Autoimmune hepatitis is a rare disease that requires chronic treatment with systemic steroids. The use of a liver-selective steroid as a low-dose, slow-release formulation is an easily understood example of the use of this invention, because the systemic effects of the chronic use of steroids are well known. These include suppression of the adrenal gland, osteoporosis, susceptibility to infection, weight gain, fluid retention, and other effects.

It is a further aspect of this invention that when used in low dose as a sustained-release formulation to achieve liver-selectivity, steroids such as prednisone may be used to treat autoimmune hepatitis without risk, or with less risk of unwanted systemic side effects.

Viral Hepatitis

All varieties of viral hepatitis, (Hepatitis A, B, C, D, E, F, G, and others) are systemic diseases, but their principal site of activity and the principal site of viral replication is in the liver. Therefore, it is desirable to concentrate a viracidal drug within the liver to enhance its efficacy. Furthermore the required cellular effects of these drugs, their frequent need in patients with impaired immune and haemopoietic systems, and other systemic effects support the desirability of liver-selective therapy. It is a further aspect of this invention that when used in low dose as a sustained-release formulation to achieve liver-selectivity, orally-administered antiviral agents from a wide range of chemical class may be used to treat viral hepatitis with less risk of unwanted systemic side effects.

Hepatic Hypoxia

Ninety to ninety-five percent of the blood flow to the liver is venous carrying less than arterial levels of oxygen. While the liver is very capable of operating at relatively low oxygen levels, any condition that reduces venous perfusion is know to reduce intrahepatic oxygen levels to hypoxic levels and thereby reduce liver function over and beyond the depressant effect of the underlying disease. Diseases associated with intrahepatic hypoxia include cirrhosis of the liver (in which portal venous flow is impeded by fibrosis and tissue damage), all forms of viral hepatitis where flow is impeded by swelling of the inflamed hepatocytes, other forms of hepatitis including alcoholic hepatitis, and congestion of the liver caused by cardiac failure and caval obstruction.

Hypoxia of any tissue in any organ causes elevation of intracellular reducing compounds such as NADPH₂ which then act to contribute to the production of free radicals. Free radicals, and in particular the hydroxy free-radical, attack phospholipid within cell membranes converting small amounts to lysophospholipid. This has the effect of increasing the permeability of the membranes allowing entry of calcium ions and other substances. The membrane damage is followed by a cascade of cellular dysfunction presenting as organ dysfunction or cell death. In the case of the heart or brain, antioxidants that act to absorb free radicals can delay the hypoxic damage including infarction, but their effect is very transitory on account of the severity of the oxygen deficit which is sufficient to cause cell death. By contrast, disease processes within the liver create moderate rather than fatal hypoxia that may last for many months albeit with diminished function of the liver.

It is a further aspect of this invention that when used in low dose as a sustained-release formulation to achieve liver-selectivity, orally-administered antioxidants from a wide range of chemical class may be used to treat diseases of the liver characterised by hypoxia. Administered in this way, a therapeutic effect may be achieved with no or minimal risk of systemic side effects.

Other Conditions

It is a further aspect of this invention that it applies to any other condition where the liver itself is the primary therapeutic target and it is desirable to concentrate a therapeutic agent within the liver.

Complementary Medicines

Modern pharmacotherapy is progressively using herbal and traditional medicines to complement the use of prescription medicines.

It is a further aspect of this invention that when used as a low-dose, sustained-release formulation, any orally-administered herbal or complementary medicine product selected because of its known or perceived ability to treat liver disease, will act as a liver-selective treatment. This claim is based on the principle that the active agent or agents of a herbal or complementary product must be absorbed into the body and pass through the portal circulation and liver in the same way as any other therapeutic agent.

Formulation for Slow-Release

There are many techniques to effect slow release of an active pharmaceutical agent from an orally-administered formulation. The present invention claims the principle of formulating a low dose of a drug with a short half-life as a slow-release formulation to produce liver selectivity, and it is intended to cover any method of slow-release formulation. These methods may include techniques designed to delay the disintegration of a capsule, tablet, or other vehicle, techniques designed to delay the solubility of a capsule, tablet or other vehicle, and techniques in which an active agent may be bound to a polymer or other large molecule such that absorption can not take place until the substance has been released from the polymer or other large molecule. The means of achieving these different methods of slow release are varied and include older methods such as layers of shellac coating, and more modern techniques with synthetic and cellulose polymers.

A slow release formulation may be designed to release an active agent over 12 hours thereby permitting twice-a-day administration, or over 24 hours permitting once-a-day administration. It is a feature of formulations releasing drug over longer periods of time that they may have more than one timed-release component to effect time coverage.

The invention will now be described with reference to the following examples. It is to be understood that the examples are provided by way of illustration of the invention and that they are in no way limiting to the scope of the invention.

### EXAMPLE

Exemplification of the Kinetic Principle of Liver-Selective Therapy

Experiments were undertaken in four dogs under general anaesthesia induced with halothane and then maintained with ketamine and xylazine. Cardiovascular status was monitored by measurement of heart rate and blood pressure and by measurement of arterial blood gases. Ventilation was assisted to maintain blood gases within physiological limits. A catheter was placed in the femoral artery to permit sampling of arterial blood. After laparotomy, a catheter was placed in a mesenteric vein and advanced to the portal vein to permit sampling of portal venous blood samples.

Propranolol was administered by mouth on the evening before, and then again one hour before the study as a dose of 40 mg in granules taken from a 160 mg slow release formulation of propranolol (Cardinol; Pacific Pharmaceuticals New Zealand). Paired blood samples were then taken from systemic artery and femoral vein at 0, ½, 1, 1½, and 2, for measurement of the blood concentration of propranolol. The animals were sacrificed at the end of he experiment.

Results are displayed in the Table. The concentration in systemic blood was generally below the level of detection (<5 ug/ml).

Kinetic Studies

| Propranolol Concentration ug/ml | | | | |
|---|---|---|---|---|
| Dog 1 | Dog 2 | Dog 3 | Dog 4 | Mean |

Portal Vein

| Baseline | | <5 | 28.8 | 21 | 36 |
|---|---|---|---|---|---|
| 30 | min | 11.5 | 11.8 | 13 | 2 |
| 60 | min | 5.8 | 10.9 | 10 | 6 |
| 90 | min | 22 | 14.4 | 10 | 5 |
| 120 | min | <5 | | 13 | 4 |

Systemic

| Baseline | | <5 | <5 | 3 | <2 |
|---|---|---|---|---|---|
| 30 | min | <5 | <5 | 2 | <2 |
| 60 | min | 6.2 | <5 | 2 | <2 |
| 90 | min | 5.8 | <5 | 2 | <2 |
| 120 | min | <5 | | 2 | <2 |

In this small series in dogs, the data indicate concentration gradients between portal and systemic vessels to provide liver selective therapy.

It is to be understood hat the invention herein above is susceptible to variations, modifications, and/or additions other than those specifically described and that the invention includes all such variations, modifications and/or additions which fall within the spirit and scope of the above description.

1. A method of treating a patient having hypercholesterolemia comprising:
orally administering to the patient at least one statin in a slow release formulation which provides a release rate which provides a first pass hepatic clearance that approaches but does not reach total hepatic clearance comprising a statin in a dose amount of from about 1 mg to about 40 mg, wherein the formulation releases the defined dose over a time period of from about 6 to about 24 hours.

2. A method of treatment of a patient suffering from portal hypertension comprising administering orally to the patient a slow release formulation of at least one beta-blocker to provide a dose-delivery rate sufficient to provide beta-blockade in the liver and portal system and less than required to provide a blood level in the peripheral circulation that has an inhibitory effect on heart rate.

3. A method according to claim 2, wherein the at least one beta-blocker comprises propranol.

4. A method according to claim 3, wherein the at least one beta-blocker comprises administered as a slow-release formulation at a dose equivalent to from 10 to 25 mg per day of propranolol.

5. The method according to claim 1, wherein the defined dose is administered once a day.

6. The method according to claim 1, wherein the at least one statin has a formula: 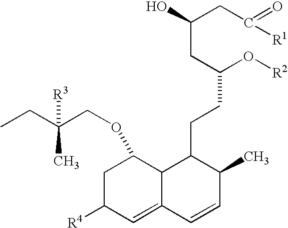
wherein:
R¹ is OR5, wherein R5 is a counter ion,
R3 is hydrogen or methyl,
R4 is chosen from hydrogen, hydroxyl, and methyl,
R2 is hydrogen, or R1 and R2 may together form a bond to provide a lactone; and
wherein the slow-release formulation provides a dose delivery rate sufficient to provide a cholesterol lowering effect in the lever and less than required to provide inhibition of systemic synthesis of ubiquinone.

7. The method according to claim 1 wherein the at least one statin is chosen from simvastatin, pravastain, mevastatin, lovastatin, fluvastatin, cerivastatin, rosuvastatin, and atrovastatin.

8. The method according to claim 7, wherein the at least one statin comprises simvastatin.

9. A method of treatment of a patient suffering from autoimmune hepatitis comprising administering to the patient at least one steroid effective in treating hepatitis wherein the at least one steroid is administered orally in a slow-release formulation providing a dose-delivery rate sufficient to provide effective steroid levels in the portal system and less than required to provide a systemic blood level to produce systemic effects.

10. A method according to claim 12, wherein the at least one steroid comprises prednisone or another equivalent corticosteroid.

11. A method of treatment of a patient suffering from hepatic hypoxia comprising orally administering to the patient at least one antioxidant in a slow release formulation at a dose-delivery rate sufficient to provide an effective blood level in the portal system and less than that required to provide blood levels in the peripheral circulation sufficient to produce a clinical or adverse effect.

12. A method of treatment of a patient suffering from a form of liver disease other than portal hypertension, autoimmune hepatitis and hepatic hypoxia comprising administering to the patient a slow release formulation of drug sufficient to achieve effective blood levels in the liver and portal venous system and less than that required to produce a clinical or adverse effect elsewhere in the body.

13. A method of treatment of a patient suffering from a form of liver disease other than portal hypertension, hypercholesterolemia, hepatitis, viral hepatitis and hepatic hypoxia comprising administering to the patient a slow release of at least one complementary medicine or herbal product sufficient to achieve effective blood levels in the liver and portal venous system and less than that required to produce clinical or adverse effects elsewhere in the body.

14. The method according to claim 1, wherein the at least one statin exhibits an aqueous solubility at room temperature of less than 5 grams per liter.

15. The method according to claim 14, wherein the at least one statin is chosen from lovastatin, simvastatin, atorvastatin, and fluvastatin.

16. The method according to claim 14, wherein the at least one statin exhibits an aqueous solubility at room temperature of less than 1 gram per liter.

17. The method according to claim 16, wherein the at least one statin is chosen from lovastatin, simvastatin, and atorvastatin.

18. The method according to claim 1, wherein the slow release formulation releases the at least one statin by a mechanism chosen from diffusion and erosion.

19. The method according to claim 1, wherein the slow release formulation is chosen from polymer-coated mini-tablets, hydrophilic matrix tablets, and mixtures thereof.

20. The method according to claim 1, wherein the dose ranges from about 1 mg to about 20 mg.

21. The method according to claim 20, wherein the dose ranges from about 1 mg to about 10 mg.
